(19) United States
(12) Patent Application Publication
(10) Pub. No.: US 2010/0216652 A1
(43) Pub. Date: Aug. 26, 2010

(54) Low Level Fluorescence Detection at the Light Microscopic Level

(75) Inventors: James H. Eberwine, Philadelphia, PA (US); Philip Haydon, Narberth, PA (US); Jai-Yoon Sul, Bensalem, PA (US); Kevin Miyashiro, Philadelphia, PA (US); Thomas Bell, Turnersville, NJ (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: THE TRUSTEES OF THE UNIVERSIT OF PENNSYLVANIA

(21) Appl. No.: 12/597,452

(22) PCT Filed: Apr. 24, 2008
(86) PCT No.: PCT/US08/05294

§ 371 Date: May 4, 2010

Related U.S. Application Data
(60) Provisional application No. 60/926,360, filed on Apr. 25, 2007.

(51) Int. Cl. C40B 30/00; C12Q 1/68; C12Q 1/02; G01N 33/53; G01N 33/68; G01N 33/48; G01N 21/64

(52) U.S. Cl. 506/7; 435/6; 435/7.92; 435/29; 436/501; 436/86; 436/94; 436/172

(57) ABSTRACT

The present invention discloses methods of removing unwanted fluorescence from a sample by photobleaching said sample to enhance detection of proteins and fragments thereof, polynucleotides and fragments thereof, and biomolecules and fragments thereof in a sample by contacting said proteins, polynucleotides and biomolecules with a fluorescent reporter, wherein said fluorescent reported comprises a fluorescent semiconductor crystal or SCN, wherein said SCN further comprises a targeting moiety.

### STATEMENT REGARDING FEDERALLY SPONSORED RESEARCH OR DEVELOPMENT

This invention was made, in part, using funds obtained from the U.S. Government (National Institutes of Health Grant No. AG9900), and the U.S. Government may therefore have certain rights in this invention.

### BACKGROUND OF THE INVENTION

Many types of biological and industrial research rely on the ability to mark or label microscopic structures (e.g. cells, subcellular organelles) in order to track their movement, differentiation, or mark the association of a variety of components within an organism or other medium. With advances in microscopy technique, both fluorescence and chromogenic methods have become widely used though fluorescence is often the end detection point for the majority of biological measurements made in diverse disciplines—including DNA sequencing, microarray chips, neuroanatomical tracing studies, immunohistochemistry, ELISAs, and functional cellular assays such as Ca²⁺ imaging and voltage sensitive dyes. Although both methods are highly sensitive, they each have limitations in detecting cellular target. For example, color development (e.g. alkaline phosphates) is an excellent approach to detect low abundance targets, but it lacks the ability to finely discriminate subcellular localization. In comparison, fluorescence microscopy lacks the sensitivity of color development predominantly because of photobleaching of target secondary fluorophores or endogenous autofluorescence of biological samples.

Traditionally favored materials for such applications have been organic dyes which can be chemically engineered to adhere to a diverse variety of cellular structures. After the dye comes into contact with the appropriate cellular structure, technicians may use light of a certain wavelength to excite the dye into fluorescence, whereby it emits radiation at a peak wavelength dictated by the chemical nature of the organic dye being used.

Unfortunately, there are several shortcomings associated with using fluorophores as reporter molecules. Most difficulties with the technique result from the extremely limited absorptive and emissive capabilities of organic dyes. For example, the peak emission of organic dyes cannot be altered—each dye corresponds to a different molecule with a different pre-set emission wavelength (color) that is set by nature. Therefore, applications that make use of light frequencies that do not correspond to the emission peaks of preexisting organic dyes cannot be performed. In addition, organic fluorescent dyes have a narrow absorption pattern and not always in convenient regions of the spectrum, making the excitation of various organic dyes challenging and costly. Organic fluorescent dyes also exhibit uneven absorption and emission peaks and tend to produce ‘shoulders’ in the geometry of their emission and absorption peaks, a major disadvantage in applications that require Gaussian type emission patterns to work correctly. One of the most problematic aspects of organic fluorescent dyes is that of stability. The lifetime of organic dyes varies but is generally low relative to that of other tagging methodologies. In addition, all fluorescent dyes bleach over time upon observation. Oxygen radicals form as a side product of the photochemistry of fluorescence, which react with the dyes and destroy them. Photobleaching is especially problematic with confocal microscopy due to the high intensity of the laser illumination.

When using fluorescent probes in biological tissue, a primary problem is minimizing fluorescence noise in order to maximize signal detection. The first issue of detection is simply one of sensitivity as a result of the relative abundance of the target molecule. Target molecules present in abundance are usually readily detectable; however molecules present at small numbers in the tissue can limit the usefulness of the technique.

Second, detection of fluorescent signal is hampered by autofluorescence. Autofluorescence is the fluorescence of substances other than the fluorophore of interest. Biological autofluorescence occurs because cells contain molecules which become fluorescent when excited by UV/V is radiation of suitable wavelength. This fluorescence emission, arising from endogenous fluorophores, is an intrinsic property of cells and is called auto-fluorescence to distinguish it from fluorescent signals obtained by adding exogenous fluorophores. The majority of cell auto-fluorescence originates from mitochondria and lysosomes. Together with aromatic amino acids and lipo-pigments, the most important endogenous fluorophores are pyridinic (NADPH) and flavin coenzymes. In tissues, the extracellular matrix often contributes to the auto-fluorescence emission more than the cellular component, because collagen and elastin have, among the endogenous fluorophores, a relatively high quantum yield.

Third, detection of a specific signal can be impeded by background fluorescence, a result of non-specific binding of an exogenously applied fluorescent probe to a tissue sample.

Similar problems are encountered when labelling DNA with fluorescent tags. Specifically, there are two main drawbacks of the use of DNA staining agents. The first is a decrease of fluorescence over time (photobleaching), however, in this case, the release of free radicals induce cleavage of the double-stranded DNA molecule. Although the duration of fluorescence can be extended by reducing light intensity and/or using oxygen radical scavengers, dynamic studies of DNA-protein interactions require high illumination intensity and long observation times to achieve both spatial and temporal resolutions. The second drawback is that the presence of these dyes results in changes in the electrostatic, structural and mechanical properties of DNA which are likely to modify its interaction with proteins. Enzymatic inhibition has been reported for restriction endonucleases (Shäfer B., et al., 2000, Single Mol. 1:33-40; Meng X., et al., 1996, J. Biomol. Struct. Dyn. 13:945-951) or exonucleases (Matsuura S., et al., 2001, Nucleic Acids Res. 29:E79). Moreover, these dyes are flushed away from DNA under sodium and magnesium concentrations consistent with enzymatic activity (Liu Y. Y., et al., 2004, J. Chem. Phys. 121:4302-4309). These limits constrain the use of this labeling method for DNA-protein interaction studies.

Fluorescent semiconductor nanocrystals (SCN), also known as quantum dots (QD), are nanometer-sized particles composed of a heavy metal core, such as cadmium selenium or cadmium telluride, with an intermediate unreactive zinc sulfide shell. SCN can also comprise a customized outer coating of different bioactive molecules tailored to a specific application. The composition and very small size of SCN (2-10 nm) gives them unique and very stable fluorescent optical properties and dictates the emission wavelength through quantum confinement. These optical properties are readily tunable by changing their physical composition or size. The photochemical properties of SCN allow selective fluorescent tagging of proteins similar to classical immunocytochemistry. Additionally, the use of SCN is associated with minimal photobleaching and a much higher signal-to-noise ratio. Their broad absorption spectra but very narrow emission spectra allows multiplexing of many SCN of different colors in the same sample, something that cannot be achieved with traditional fluorophores.

The small size of SCN particles results in large but specific energy jumps between the energy band gaps of excited electron-hole pairs in the semiconductor core. This effect results in scaled changes of absorption and emission wavelengths as a function of particle size, so that small changes in the radius of SCN translate into very distinct changes in color (Arya et al., 2005, Biochem Biophys Res Commun 329:1173-1177; Vanmaekelbergh and Liljeroth, 2005, Chem Soc Rev 34:299-312). SCN with diameters ranging from 6.5-5.5 nm emit in the red range of the visible spectrum (620-750 nm), SCN with diameters of 4.0 nm emit in the yellow range (570-590 nm), SCN with diameters of 3.0 nm emit in the green range of the visible spectrum (495-570 nm), and SCN with diameters ranging from 2.5-2 nm emit in the blue range of the visible spectrum (450-495 nm). This physical property represents another major advantage over traditional organic fluorophores that in general require distinct chemistries to produce different colors. For biological applications, SCN can be chemically functionalized to target proteins at high ligand-receptor densities. Recent work has shown that, at least in some cellular systems, SCN conjugated with natural ligands are readily internalized into cells, do not interfere with intracellular signaling, and are nontoxic (Chan et al., 2002, Curr Opin Biotechnol 13:40-46; Murphy, 2002, Optical sensing with s. Anal Chem 74:520A-526A; Jain, 2003, Expert Rev Mol Diagn 3:153-161; Watson et al., 2003, Biotechniques 34:296-300. 302-303; West and Halas, 2003, Annu Rev Biomed Eng 5:285-292).

A variety of well known techniques are used to detect cellular proteins, polynucleotides, and other biomolecules of interest (e.g. immunohistochemistry, in situ hybridization, Western, Northern and Southern blotting, microarray, ELISA, PCR and RT-PCR). Several different visualization methods, including fluorescence, chromogens, metal beads and isotopes, are commonly used. With advances in microscopy technique, both fluorescence and chromogenic methods have become widely used. Although both methods are highly-sensitive, they each have limitations in detecting cellular targets. For example, color development (eg. alkaline phosphatase) is an excellent approach to detect low abundance targets, but lacks the ability to finely discriminate subcellular localization. In comparison, fluorescence microscopy lacks the sensitivity of color development predominantly due to photobleaching of target secondary fluorophores or endogenous autofluorescence of biological samples.

Thus there has been a long standing need in the art for a method that allows both highly sensitive detection and localization of fluorophores in biological samples. The present invention meets this need.

### SUMMARY OF THE INVENTION

One embodiment of the invention comprises a method of detecting a target molecule in a biological sample, the method comprising contacting said target molecule with a fluorescent semiconductor nanocrystal (SCN), photobleaching said sample to reduce unwanted fluorescence, and detecting said SCN, wherein said SCN comprises a modification comprising a targeting moiety. In one aspect, the biological sample is selected from a tissue, a cell, a biopsy, and a body sample. In another aspect, the SCN is water soluble. In a further aspect, the targeting moiety specifically binds a target molecule. In a further aspect, the method comprises an immunoassay selected from the group consisting of Western blot, ELISA, immunopercipitation, immunohistochemistry, immunofluorescence, radioimmunoassay, dot blotting, and FACS. In yet another aspect, the method comprises a nucleic acid assay selected from the group consisting of a Northern blot, a Southern blot, in situ hybridization, a PCR assay, an RT-PCR assay, a probe array, a gene chip, and a microarray.

Another embodiment of the invention comprises a method of detecting a protein moiety in a biological sample, the method comprising contacting the protein with a fluorescent semiconductor nanocrystal (SCN), photobleaching said sample to reduce unwanted fluorescence, and detecting the fluorescent SCN, and wherein the SCN comprises a modification comprising a targeting moiety. In one aspect, the biological sample is selected from a tissue, a cell, a biopsy, and a body sample. In another aspect, the SCN is water soluble. In yet another aspect, the targeting moiety specifically binds to said protein. In a preferred aspect, the targeting moiety comprises an antibody directed against said protein, or fragment thereof. In still other aspects of the invention, the method comprises an immunoassay selected from the group consisting of Western blot, ELISA, immunopercipitation, immunohistochemistry, immunofluorescence, radioimmunoassay, dot blotting, and FACS. In one preferred aspect, the SCN is conjugated to streptavidin. In another preferred aspect, the SCN is conjugated to a secondary antibody comprising the F(ab')₂ fragment of affinity purified antibodies cross adsorbed against serum proteins from a mammal. In a more preferred aspect, the mammal is selected from the group consisting of a human, a rat, a mouse, a rabbit, and a goat. In another aspect, the SCN is conjugated to a secondary antibody comprising the. F(ab')₂ fragment of affinity purified antibodies cross adsorbed against serum proteins from a non-mammal. In a preferred aspect, the non-mammal is a chicken.

In one aspect, the SCN emits light with a characteristic wavelength of 450-495 nm. In another aspect, the SCN emits light with a characteristic wavelength of 495-570 nm. In yet another aspect, the SCN emits light with a characteristic wavelength of 570-590 nm. In a further aspect, the SCN emits light with a characteristic wavelength of 590-620 nm. In still another aspect, the SCN emits light with a characteristic wavelength of 620-750 nm.

In another embodiment, the method of the invention comprises a method of detecting a polynucleotide moiety in a biological sample, the method comprising contacting the polynucleotide with a fluorescent SCN, photobleaching the sample to reduce unwanted fluorescence, and detecting the SCN, wherein then comprises a modification comprising a targeting moiety.

In one aspect, the biological sample is selected from a tissue, a cell, a biopsy, and a body sample. In another aspect, the SCN is water soluble. In still another aspect, the targeting moiety specifically binds to said polynucleotide moiety. In a further aspect, the method of the invention comprises detection of the polynucleotide using a nucleic acid assay selected from the group consisting of a Northern blot, a Southern blot, in situ hybridization, a PCR assay, an RT-PCR assay, a probe array, a gene chip, and a microarray.

In another embodiment of the invention, the method of the invention comprises a method of detecting a biomolecule moiety of interest in a biological sample, the method comprising contacting the biomolecule with a fluorescent SCN, photobleaching the sample to reduce unwanted fluorescence, and detecting the SCN, wherein the SCN comprises a modification comprising a targeting moiety.

In one aspect, the targeting moiety specifically binds the biomolecule of interest. In another aspect, the method comprises an immunoassay selected from the group consisting of Western blot, ELISA, immunopercipitation, immunohistochemistry, immunofluorescence, radioimmunoassay, dot blotting, and FACS. In yet another aspect, the method comprises a nucleic acid assay selected from the group consisting of a Northern blot, a Southern blot, in situ hybridization, a PCR assay, an RT-PCR assay, a probe array, a gene chip, and a microarray.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1D, is a series of charts comparing the signal intensity of Alexa 488 and Qdot-565 before and after photobleaching. FIG. 1A illustrates the emission spectral signature for Alexa 488 over 520 to 580 nm wavelength before photobleaching. FIG. 1B illustrates the emission spectral signature for Alexa 488 over 520 to 580 nm wavelength after photobleaching. FIG. 1C illustrates the emission spectral signature for Qdot 565 over 520 to 580 nm wavelength before photobleaching. FIG. 1D illustrates the emission spectral signature for Qdot 565 over 520 to 580 nm wavelength after photobleaching.

FIG. 2 is a chart illustrating spectal scanning before and after photobleaching. The solid line shows the emission spectrum resulting from 458 nm excitation and the dotted line shows the remaining emission spectrum after the photobleaching protocol.

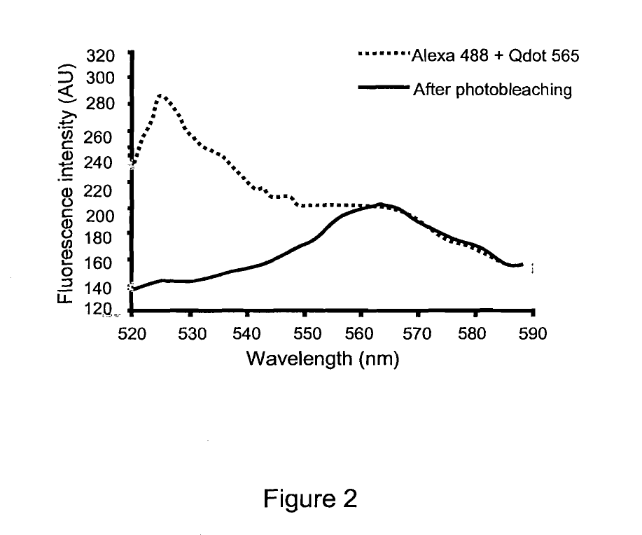

FIG. 3, comprising FIG. 3A through FIG. 3D, is a series of photomicrographs depicting ISH of cultured hippocampal neurons with KCNMA1. FIG. 3A depicts a highly autofluorescencing sample before photobleaching. The dotted square region depicted in FIG. 3A and FIG. 3B was subjected to photobleaching. FIG. 3B illustrates that most of the autofluorescence was abolished after photobleaching. FIG. 3C depicts ISH of cultured hippocampal neurons with KCNMA1 probe with autofluorescence. FIG. 3D depicts the same section after photobleaching.

### DETAILED DESCRIPTION OF THE INVENTION

The present invention is related to the discovery of a novel method for removing unwanted fluorescence from a biological sample by applying a full spectral laser scan to the sample (photobleaching), thereby enhancing detection of a photobleaching-resistant fluorophore bound to a protein, a polynucleotide, and/or a biomolecule of interest via semiconductor nanocrystal conjugated to a targeting moiety.

### Definitions:

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used in this specification and the appended claims, the singular forms “a”, “an”, and “the” include plural referents unless the content clearly dictates otherwise. Thus, for example, reference to “a pulmonary surfactant” includes a combination of two or more pulmonary surfactants, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

The phrase “biological sample,” as used herein, may comprise any primary isolated or cultured cell, tissue, organ or body sample.

A “body sample” is any sample comprising a cell, a tissue, or a bodily fluid in which expression of a protein, a polynucleotide, and/or a biomolecule can be detected. Examples of such body samples include but are not limited to blood, lymph, biopsies, amniotic fluid and smears. Samples that are liquid in nature are referred to herein as “bodily fluids.” Body samples may be obtained from an individual by a variety of techniques including, for example, by scraping or swabbing an area or by using a needle to aspirate bodily fluids. Methods for collecting various body samples are well known in the art. One of ordinary skill in the art will be familiar with the histological techniques and procedures used in the preparation of a biological sample for subsequent detection of a biomolecule of interest.

The phrase “biomolecule” as used herein, is intended a chemical compound that naturally occurs in living organisms. Biomolecules consist primarily of carbon and hydrogen, along with nitrogen, oxygen, phosphorus and sulfur. Other elements sometimes are incorporated but are much less common.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The phrase “nanocrystal” as used herein, refers to a crystalline material with dimensions measured in nanometers. Nanocrystals fabricated from semiconductor materials in the sub 10 nm size range are often referred to as quantum dots.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “semiconductor nanocrystal,” synonomous with the phrase “quantum dot” as used herein, is a semiconductor nanostructure that confines the motion of conduction band electrons, valence band holes, or excitons (bound pairs of conduction band electrons and valence band holes) in all three spatial directions. The confinement can be due to electrostatic potentials (generated by external electrodes, doping, strain, impurities), the presence of an interface between different semiconductor materials (e.g. in core-shell nanocrystal systems), the presence of the semiconductor surface (e.g. semiconductor nanocrystal), or a combination of these. A quantum dot, or SCN, has a discrete quantized energy spectrum. The corresponding wave functions are spatially localized within the quantum dot, but extend over many periods of the crystal lattice. A quantum dot contains a small finite number (of the order of 1-100) of conduction band electrons, valence band holes, or excitons, i.e., a finite number of elementary electric charges. One of the optical features of small excitonic quantum dots immediately noticeable to the unaided eye is coloration. While the material which makes up a quantum dot defines its intrinsic energy signature, more significant in terms of coloration is the size. The larger the dot, the redder (the more towards the red end of the spectrum) the fluorescence. The smaller the dot, the bluer (the more towards the blue end) it is. The coloration is directly related to the energy levels of the quantum dot. Quantitatively speaking, the bandgap energy that determines the energy (and hence color) of the fluoresced light is inversely proportional to the square of the size of the quantum dot.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

As used herein, a “targeting moiety” refers to a molecule that binds specifically to a molecule present on the cell surface of a target cell.

By the term “specifically binds,” as used herein, is meant a molecule, such as an antibody, which recognizes and binds to a cell surface molecule or feature, but does not substantially recognize or bind other molecules or features in a sample.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

### Description:

The present invention is related to the discovery that full spectral laser scans (photobleaching) of a biological sample reduce unwanted fluorescence, but preserve emission from photobleaching-resistant SCNs used as fluorophores to label one or more biomolecule of interest. The method of the present invention is applicable across a wide range of well known techniques used to detect protein, polynucleotide and other biomolecules of interest in a biological sample, including, but not limited to, immunohistochemistry and in situ hybridization.

Thus, for the first time, the present invention discloses a methodology that allows true single molecule detection and localization in cellular organelles with multiple emission wavelengths without having to perform electron microscopy in conjunction with immunohistochemistry.

In one embodiment, the present invention provides a method of detecting a target molecule of interest in a biological sample. A target molecule is any protein, polynucleotide, and/or biomolecule of interest. The method comprises contacting the sample with a fluorescent semiconductor nanocrystal (SCN) conjugated to a targeting moiety, wherein the targeting moiety of the SCN conjugate specifically binds to the target molecule of interest. The method further comprises photobleaching the sample by exposing it to a full spectral laser scan to remove unwanted background fluorescence and autofluorescence and detecting the remaining fluorescence, wherein the detection of fluorescence indicates that the SCN conjugate bound a target molecule of interest.

### Target Molecules

The phrase “target molecule” as used herein refers to a vast array of biomolecules present in a biological sample that can be detected using the method of the present invention. Biomolecules are a diverse class of chemical compounds that naturally occur in living organisms. Biomolecules consist primarily of carbon and hydrogen, along with nitrogen, oxygen, phosphorus and sulfur, although other elements sometimes are less commonly incorporated. One skilled in the art would appreciate that biomolecules include, but are not limited to small molecules (e.g. lipids, hormones, neurotransmitters, carbohydrates, sugars), monomers (e.g. amino acids, nucleotides, phosphate), and polymers (e.g. peptides, polypeptides, proteins, nucleic acids including RNA and DNA, and poly saccharides).

A subclass of biomolecule, proteins are large organic compounds comprising linearly arranged amino acids linked by peptide bonds. There is some ambiguity between the usage of the words protein, polypeptide, and peptide. Protein is generally used to refer to the complete biological molecule in a stable conformation, while peptide is generally reserved for a short amino acid oligomers often lacking a stable 3-dimensional structure. However, the boundary between the two is ill-defined and usually lies near 20-30 residues. Polypeptide can refer to any single linear chain of amino acids, usually regardless of length, but often implies an absence of a single defined conformation.

Polynucleotides include, but are not limited to, all nucleic acid sequences which are obtained by any means available in the art, including, without limitation, recombinant means, comprising both RNA and DNA and nucleic acids.

It is understood by one skilled in the art that the method of the present invention is not limited by the origin, sequence or composition of the target molecule being detected in the biological sample.

### Semiconductor Nanocrystals (SCN)

The method of the present invention utilizes semiconductor nanocrystals (SCN) as ultrasensitive nonisotopic reporters of biomolecules in vitro and in vivo. SCN are attractive fluorescent tags for biological molecules due to their large quantum yield and photostability. As such, SCN overcome many of the limitations inherent to the organic dyes used as conventional fluorophores. SCN range from 2 nm to 10 nm in diameter, contain approximately 500-1000 atoms of materials such as cadmium and selenide, and fluoresce with a broad absorption spectrum and a narrow emission spectrum.

A water-soluble luminescent SCN, which comprises a core, a cap and a hydrophilic attachment group is well known in the art and commercially available (e.g. Quantum Dot Corp. Hayward, Calif.; Invitrogen, Carlsbad, Calif.; U.S. Pat. No. 7,192,785; U.S. Pat. No. 6,815,064). The “core” comprises a nanoparticle-sized semiconductor. While any core of the IIB VIB, IIIB VB or IVB-IVB semiconductors can be used in the context of the present invention, the core must be such that, upon combination with a cap, a luminescence results. A IIB VIB semiconductor is a compound that contains at least one element from Group IEB and at least one element from Group VIB of the periodic table, and so on. Preferably, the core is a IIB VIB, IIIB VB or IVB-IVB semiconductor that ranges in size from about 1 nm to about 10 nm. The core is more preferably a IIB VIB semiconductor and ranges in size from about 2 nm to about 5 nm. Most preferably, the core is CdS or CdSe. In this regard, CdSe is especially preferred as the core.

The “cap” is a semiconductor that differs from the semiconductor of the core and binds to the core, thereby forming a surface layer on the core. The cap must be such that, upon combination with a given semiconductor core, a luminescence results. The cap should passivate the core by having a higher band gap than the core. In this regard, the cap is preferably a IIB VIB semiconductor of high band gap. More preferably, the cap is ZnS or CdS. Most preferably, the cap is ZnS. In particular, the cap is preferably ZnS when the core is CdSe or CdS and the cap is preferably CdS when the core is CdSe.

The “attachment group” as used herein, refers to any organic group that can be attached, such as by any stable physical or chemical association, to the surface of the cap of the SCN. In one embodiment, the attachment group can render the SCN water-soluble without rendering the SCN no longer luminescent. Accordingly, the attachment group comprises a hydrophilic moiety. Preferably, the attachment group enables the hydrophilic SCN to remain in solution for at least about one hour. More preferably the attachment group enables the hydrophilic SCN to remain in solution for at least about one day. Even more preferably, the attachment group allows the hydrophilic SCN to remain in solution for at least about one week, most preferably for at least about one month. Desirably, the attachment group is attached to the cap by covalent bonding and is attached to the cap in such a manner that the hydrophilic moiety is exposed. Preferably, the hydrophilic attachment group is attached to the SCN via a sulfur atom. More preferably, the hydrophilic attachment group is an organic group comprising a sulfur atom and at least one hydrophilic attachment group. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. The organic group of the hydrophilic attachment group of the present invention is preferably a C₁ C₆ alkyl group or an aryl group, more preferably a C₁ C₆ alkyl group, even more preferably a C₁ C₃ alkyl group. Therefore, in a preferred embodiment, the attachment group of the present invention is a thiol carboxylic acid or thiol alcohol. More preferably, the attachment group is a thiol carboxylic acid. Most preferably, the attachment group is mercaptoacetic acid.

Accordingly, a preferred embodiment of a water-soluble SCN is one that comprises a CdSe core ranging from 2-10 nm in size, a ZnS cap and an attachment group. Another preferred embodiment of a water soluble SCN is one that comprises a CdSe core, a ZnS cap and the attachment group mercaptoacetic acid.

In another embodiment, the present invention also provides a composition comprising a water-soluble SCN as described above and an aqueous carrier. Any suitable aqueous carrier can be used in the composition. Desirably, the carrier renders the composition stable at a desired temperature, such as room temperature, and is of an approximately neutral pH. Examples of suitable aqueous carriers are known to those of ordinary skill in the art and include saline solution and phosphate-buffered saline solution (PBS).

### Targeting Moieties

The SCN is directed to a target molecule by linking a targeting moiety to the SCN. A targeting moiety may be an antibody, a naturally-occurring ligand for a receptor or functional derivatives thereof, a vitamin, a small molecule mimetic of a naturally-occurring ligand, a peptidomimetic, a polypeptide or aptamer, or any other molecule provided it binds specifically to a cell surface molecule, or a fragment thereof. Any cell surface molecule may be targeted provided binding of the targeted SCN is specific. Cell surface molecules that may be targeted include, but are not limited to, cell adhesion molecules (CAM), Glycosylphosphatidylinisotol (GPI)-anchored proteins, receptors, including but not limited to hormone receptors (e.g., epidermal growth factor receptor), sugar receptors (e.g., mannose receptor and lectin receptor), glutamate receptor mGluR5, gamma c cytokine receptor, TGF-β receptor, neurotransmitter and neuropeptide receptors, and ion channels, comprising voltage- and ligand-gated ion channel.

### Targeting Moiety-Antibodies

When the antibody used as a targeting moiety in the compositions and methods of the invention is a polyclonal antibody (IgG), the antibody is generated by inoculating a suitable animal with the targeted cell surface molecule. Antibodies produced in the inoculated animal which specifically bind to the cell surface molecule are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1988, In: Antibodies, A Laboratory Manual, Cold Spring Harbor, N.Y.).

Monoclonal antibodies directed against a full length targeted cell surface molecule or fragments thereof may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a targeted cell surface molecule, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with targeted cell surface molecule epitopes. These antibodies are capable of binding to the targeted cell surface molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Nematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the targeted cell surface molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1988, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as targeting moieties in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Samples may need to be modified in order to render the target molecule antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

One of skill in the art will appreciate that it may be desirable to detect more than one protein of interest in a biological sample. Therefore, in particular embodiments, at least two antibodies directed to two distinct proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

### Targeting Moieties—Protein, Peptide, and Polypeptide

Other types of targeting moieties useful in the invention may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the a-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the a-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a targeting moiety, a peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and other peptide targeting moieties may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### SCN-Targeting Moiety Conjugates

In one embodiment, the present invention provides a conjugate comprising a water-soluble SCN as described above and a targeting moiety, wherein the targeting moiety is attached to the SCN via the hydrophilic attachment group. The targeting moiety should not render the SCN water-insoluble. Preferably, the targeting moiety is a protein, a fragment of a protein, or a nucleic acid. Use of the phrase “protein or a fragment thereof” is intended to encompass a protein, a glycoprotein, a polypeptide, a peptide, and the like, whether isolated from nature, of viral, bacterial, plant or animal (e.g., mammalian, such as human) origin, or synthetic. A preferred protein or fragment thereof for use as a targeting moiety in the present inventive conjugate is an antigen, an epitope of an antigen, an antibody, or an antigenically reactive fragment of an antibody. Use of the phrase “nucleic acid” is intended to encompass DNA and RNA, whether isolated from nature, of viral, bacterial, plant or animal (e.g., mammalian, such as human) origin, synthetic, single-stranded, double-stranded, comprising naturally or nonnaturally occurring nucleotides, or chemically modified. A preferred nucleic acid is a single-stranded oligonucleotide comprising a stem and loop structure and the hydrophilic attachment group is attached to one end of the single-stranded oligonucleotide.

The targeting moiety can be attached, such as by any stable physical or chemical association, to the hydrophilic attachment group of the water-soluble luminescent SCN directly or indirectly by any suitable means. Desirably, the targeting moiety is attached to the attachment group directly or indirectly through one or more covalent bonds. If the targeting moiety is attached to the hydrophilic attachment group indirectly, the attachment preferably is by means of a “linker.” Use of the term “linker” is intended to encompass any suitable means that can be used to link the targeting moiety to the attachment group of the water-soluble luminescent SCN. The linker should not render the water-soluble luminescent SCN water-insoluble and should not adversely affect the luminescence of the SCN. Also, the linker should not adversely affect the function of the attached targeting moiety. If the conjugate is to be used in vivo, desirably the linker is biologically compatible.

For example, if the attachment group is mercaptoacetic acid and a nucleic acid targeting moiety is being attached to the attachment group, the linker preferably is a primary amine, a thiol, streptavidin, neutravidin, biotin, or a like molecule. If the attachment group is mercaptoacetic acid and a protein targeting moiety or a fragment thereof is being attached to the attachment group, the linker preferably is strepavidin, neutravidin, biotin, or a like molecule. In accordance with the invention, the linker should not contact the protein targeting moiety or a fragment thereof at an amino acid which is essential to the function or activity of the attached protein. Crosslinkers, such as intermediate crosslinkers, can be used to attach a targeting moiety to the attachment group of the water-soluble SCN. Ethyl-3-(dimethylaminopropyl) carbodiimide (EDAC) is an example of an intermediate crosslinker. Other examples of intermediate crosslinkers for use in the present invention are known in the art. See, for example, Bioconjugate Techniques (Academic Press, New York, (1996)).

Catalytic crosslinkers also can be used to attach a b targeting moiety to the attachment group of the water-soluble SCN. Catalytic crosslinkers effect direct attachment of the targeting moiety to the attachment group. Examples of catalytic crosslinkers are also known in the art. See, for example, Bioconjugate Techniques (1996), supra.

Attachment of a targeting moiety to the attachment group of the water-soluble SCN also can be effected by a bi-functional compound as is known in the art. See, for example, Bioconjugate Techniques (1996), supra.

In those instances where a short linker could cause steric hindrance problems or otherwise affect the functioning of the targeting moiety, the length of the linker can be increased, e.g., by the addition of from about a 10 to about a 20 atom spacer, using procedures well-known in the art (see, for example, Bioconjugate Techniques (1996), supra). One possible linker is activated polyethylene glycol, which is hydrophilic and is widely used in preparing labeled oligonucleotides.

Accordingly, a preferred conjugate in accordance with the present invention is a conjugate comprising a CdSe core between 2-10 nm, a ZnS cap, a hydrophilic attachment group and a targeting moiety. Another preferred conjugate in accordance with the present invention is a conjugate comprising a CdSe core, a ZnS cap, a mercaptoacetic acid attachment group and a targeting moiety. An especially preferred conjugate comprises a CdSe core ranging from 2-8 nm, a ZnS coating of about 1 nm, a mercaptoacetic acid attachment group and a targeting moiety.

Preferably, the SCN of the conjugate is first derivatized with streptavidin according to well-known cross-linking methods and then conjugated to the 5′ biotin group, preferably at a 1:1 molar ratio.

Thus, in another embodiment, the present invention also provides a composition comprising a conjugate as described above and an aqueous carrier. Any suitable aqueous carrier can be used in the composition. Desirably, the carrier renders the composition stable at a desired temperature, such as room temperature, and is of an approximately neutral pH. Examples of suitable aqueous carriers are known to those of ordinary skill in the art and include saline solution and PBS.

In view of the above, the present invention further provides a method of obtaining a water-soluble SCN as described. The method comprises reacting a SCN as described above in a nonpolar organic solvent with a first aqueous solution comprising an attachment group; adding a second aqueous solution of about neutral pH and mixing; and extracting an aqueous layer, thereby obtaining a water-soluble SCN. Preferably, the nonpolar organic solvent is chloroform and the attachment group is mercaptoacetic acid.

The present invention also provides a method of making a conjugate comprising a water-soluble SCN and a targeting moiety as described above. Where the targeting moiety is to be directly attached to the attachment group of the SCN, the method comprises contacting a water-soluble SCN as described above with a biomolecule, which can directly attach to the attachment group on the cap of the water-soluble SCN; and isolating the conjugate. Preferably, the targeting moiety is a protein or a fragment thereof or a nucleic acid. In one embodiment of the method of directly attaching the targeting moiety to the attachment group, the attachment group is mercaptoacetic acid and the targeting moiety is a protein. In another embodiment of the direct attachment method, the SCN and the targeting moiety e are contacted in the presence of a catalytic crosslinker.

Where the targeting moiety is to be indirectly attached to the attachment group of the water-soluble SCN, the present invention provides a method comprising contacting a water-soluble semiconductor SCN as described above with a linker, which can attach to the attachment group and the targeting moiety; isolating the water-soluble SCN to which is attached a linker; contacting the water soluble SCN to which is attached a linker with a targeting moiety; and isolating the conjugate.

Alternatively, the method comprises contacting a targeting moiety with a linker, which can attach to the attachment group and the targeting moiety; isolating the targeting moiety to which is attached a linker; contacting the targeting moiety to which is attached a linker with a water-soluble SCN; and isolating the conjugate. In one embodiment of the method of indirectly attaching the targeting moiety to the attachment group, the linker is a primary amine or streptavidin, the attachment group is mercaptoacetic acid and the targeting moiety is a nucleic acid.

In another embodiment of the method of indirectly attaching the targeting moiety to the attachment group, the method comprises contacting a water-soluble SCN with an intermediate crosslinker or a bifunctional molecule, either one of which can attach to the attachment group and the targeting moiety; isolating the water-soluble SCN to which is attached the intermediate crosslinker or the bifunctional molecule; contacting the water-soluble SCN to which is attached the intermediate crosslinker or the bifunctional molecule with a targeting moiety; and isolating the conjugate.

Alternatively, the method comprises contacting a targeting moiety with an intermediate crosslinker or a bifunctional molecule, either one of which can attach to the attachment group and the targeting moiety; isolating the targeting moiety to which is attached the intermediate crosslinker or the bifunctional molecule; contacting the targeting moiety to which is attached the intermediate crosslinker or the bifunctional molecule with a water-soluble SCN; and isolating the conjugate. An example of such an embodiment is a method employing mercaptoacetic acid as the attachment group, a protein or a fragment thereof as the targeting moiety, and EDAC as the intermediate crosslinker.

### Photobleaching

Photobleaching is the photochemical destruction of a fluorophore by photon induced chemical damage and chemical modification. Upon transition from an excited singlet state to the excited triplet state, fluorophores may interact with another molecule to produce irreversible covalent modifications. The triplet state is relatively long-lived with respect to the singlet state, thus allowing excited molecules a much longer timeframe to undergo chemical reactions with components in the environment.

The average number of excitation and emission cycles that occur for a particular fluorophore before photobleaching is dependent upon the molecular structure and the local environment. Some fluorophores bleach quickly after emitting only a few photons, while others that are more robust can undergo thousands or millions of cycles before bleaching. For a typical fluorochrome, i.e. fluorescein, the quantum yield for photobleaching of at medium to high illumination intensity dictates that an average molecule will emit between 30 to 40 thousand photons during its useful lifetime (before becoming permanently disabled). In addition, the number of excitation and emission cycles is constant for a given fluorophore regardless of how the excitation energy is delivered, either in discrete pulses or through continuous illumination. Therefore, reducing the excitation light level by using neutral density filters does not prevent photobleaching, it merely reduces the rate.

An important class of photobleaching event is photodynamic, meaning the interaction of the fluorophore with a combination of light and oxygen. Reactions between fluorophores and molecular oxygen permanently destroy fluorescence and yield a free radical singlet oxygen species that can chemically modify other molecules in living cells. The amount of photobleaching due to photodynamic events is a function of the molecular oxygen concentration and the proximal distance between the fluorophore, oxygen molecules, and other cellular components. Photobleaching can be reduced by limiting the exposure time of fluorophores to illumination or by lowering the excitation energy. However, these techniques also reduce the measurable fluorescence signal.

Semiconductor nanocrystals are extremely resistant to photobleaching. The method of the present invention exploits this resistance by applying a full spectral laser scan to a biological sample for 2 seconds, effectively destroying any fluorescence derived from sources other than SCNs (i.e. background and autofluorescence). This method boosts the sensitivity of fluorescence confocal microscopy to the point where a single SCN is detectable in a biological sample.

Without wishing to be bound by any particular theory, it will be appreciated by one skilled in the art that the autofluorescence present in any given biological sample is both a feature inherent to the sample itself, as well as a product of the histochemical processing (e.g. fixation) applied to the biological sample. The method of the present invention utilizes two (2) seconds of full-spectral laser scanning to photobleach background and autofluoresence in cultured hippocampal neurons, but contemplates routine optimization of the method to empirically determine the best photobleaching parameters for different biological samples.

### Detection Using SCN as Fluorophores

Any methods available in the art for identification or detection of a protein, a polynucleotide, or a biomolecule of interest are encompassed herein. Methods for detecting a molecule of interest (herein known as a target molecule) comprise any method that determines the quantity or the presence of the target molecule either at the nucleic acid or protein level.

The invention should not be limited to any one method of protein, nucleic acid or biomolecule detection method recited herein, but rather should encompass all known or heretofor unknown methods of detection as are, or become, known in the art.

In one embodiment, the target molecule of interest is detected at the protein level. The method comprises contacting the sample with a SCN-targeting moiety conjugate as described above, wherein the targeting moiety of the conjugate specifically binds to the protein target molecule; photobleaching the sample to remove unwanted fluorescence, and detecting residual fluorescence, wherein the detection of fluorescence indicates that the conjugate bound to a protein in the sample. Preferably, the targeting moiety of the conjugate is an antibody.

In one aspect, the method of the invention is used to detect a protein of interest in a biological sample using methods well known in the art that include, but are not limited to, western blots, ELISA, immunoprecipitation, immunofluorescence, flow cytometry, immunocytochemistry techniques.

In another embodiment, the target molecule of interest is detected at the nucleic acid level. The method comprises contacting the sample with a SCN-targeting moiety conjugate as described above, wherein the targeting moiety of the conjugate specifically binds to the nucleic acid; photobleaching the sample to remove unwanted fluorescence, and detecting residual fluorescence, wherein the detection of fluorescence indicates that the conjugate bound to the nucleic acid in the sample. Preferably, the targeting moiety of the conjugate is a nucleic acid. Alternatively, the targeting moiety of the conjugate is a protein or a fragment thereof that binds to a nucleic acid, such as a DNA binding protein.

Nucleic acid-based techniques for assessing expression are well known in the art and include, for example, Northern and Southern blots, nucleic acid amplification, including detecting mRNA in a biological sample by RT-PCR. Many expression detection methods use isolated RNA. Any RNA isolation technique that does not select against the isolation of mRNA can be utilized for the purification of RNA from biological samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target molecule, for example, a nucleotide transcript or a protein encoded by or corresponding to a target molecule. Probes can be synthesized by one of skill in the art, or derived from appropriate biological preparations. As contemplated in the present invention, a probe may be used as targeting moiety and conjugated to an SCN of a particular size. Examples of molecules that can be used as probes include, but are not limited to, RNA, DNA, proteins, antibodies, and organic molecules.

Isolated mRNA can be detected in hybridization or amplification assays that include, but are not limited to, northern blot, polymerase chain reaction and probe arrays. One method for the detection of mRNA levels involves contacting the isolated mRNA with a nucleic acid targeting moiety (probe) that can hybridize to the target molecule mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a target molecule. Hybridization of an mRNA with the probe indicates that the target molecule in question is being expressed.

The mRNA can be immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

Expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of target molecule expression may also comprise using nucleic acid probes in solution.

In one embodiment of the invention, microarrays are used to detect target molecule expression in a biological sample. Microarrays are particularly well suited for this purpose because of the reproducibility between trials. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

Nucleic acids which code for a target molecule can be placed in an array on a substrate, such as on a chip (e.g., DNA chip or microchips). These arrays also can be placed on other substrates, such as microtiter plates, beads or microspheres. Methods of linking nucleic acids to suitable substrates and the substrates themselves are described, for example, in U.S. Pat. Nos. 5,981,956; 5,922,591; 5,994,068 (Gene Logic's Flow-thru ChipO Probe ArraysO); U.S. Pat. Nos. 5,858,659, 5,753,439; 5,837,860 and the FlowMetrix technology (e.g., microspheres) of Luminex (U.S. Pat. Nos. 5,981,180 and 5,736,330).

There are two preferred methods to make a nucleic acid array. One is to synthesize the specific oligonucleotide sequences directly onto the solid-phase in the desired pattern (Southern, 1994, Nucl. Acids Res., 22: 1368-73; Maskos, 1992, Nucl. Acids Res., 20: 1679-84; Pease, 1994, Proc. Natl. Acad. Sci., 91: 5022-6; and U.S. Pat. No. 5,837,860) and the other is to presynthesize the oligonucleotides in an automated DNA synthesizer and then attach the oligonucleotides onto the solid-phase support at specific locations (Lamture, 1994, Nucl. Acids Res., 22: 2121; Smith, 1994, Nucl. Acids Res., 22: 5456 64. In the first method, the efficiency of the coupling step of each base affects the quality and integrity of the nucleic acid molecule array.

A second, more preferred method for nucleic acid array synthesis utilizes an automated DNA synthesizer for DNA synthesis. The controlled chemistry of an automated DNA synthesizer allows for the synthesis of longer, higher quality DNA molecules than is possible with the first method. Also, the nucleic acid molecules synthesized can be purified prior to the coupling step. The nucleic acids can be attached to the substrate as described in U.S. Pat. No. 5,837,860.

Thus, for example, covalently immobilized nucleic acid molecules may be used to detect specific PCR products by hybridization where the capture probe is immobilized on the solid phase or substrate (Ranki, 1983, Gene, 21: 77-85; Keller, 1991, Clin. Microbiol., 29: 638-41; Urdea, 1987, Gene, 61: 253-64). A preferred method would be to prepare a single-stranded PCR product before hybridization. A biological sample that is suspected to contain the target molecule, or an amplification product thereof, would then be exposed to the solid-surface and permitted to hybridize to the bound oligonucleotide.

The methods of the present invention do not require that the target nucleic acid contain only one of its natural two strands. Thus, the methods of the present invention may be practiced on either double-stranded DNA (dsDNA), or on single-stranded DNA (ssDNA) obtained by, for example, alkali treatment of native DNA. The presence of the unused (non-template) strand does not affect the reaction.

Where desired, however, any of a variety of methods can be used to eliminate one of the two natural stands of the target DNA molecule from the reaction. Single-stranded DNA molecules may be produced using the ssDNA bacteriophage, M13 (Messing, 1983, Meth. Enzymol., 101: 20-78; see also, Sambrook, 2001, Molecular Cloning: A Laboratory Manuel, 3^{rd} ed. (Cold Spring Harbor Laboratory Press, Cold Spring Harbor, N.Y.).

Several alternative methods can be used to generate single-stranded DNA molecules. For example, Gyllensten, 1988, Proc. Natl. Acad. Sci. U.S.A., 85: 7652-6 and Mihovilovic, 1989, BioTechniques, 7: 14-6 describe a method, termed “asymmetric PCR,” in which the standard “PCR” method is conducted using primers that are present in different molar concentrations.

Other methods have also exploited the nuclease resistant properties of phosphorothioate derivatives in order to generate single-stranded DNA molecules (U.S. Pat. No. 4,521,509; Sayers, 1988, Nucl. Acids Res., 16: 791-802; Eckstein, 1976, Biochemistry 15: 1685-91; Ott, 1987, Biochemistry 26: 8237-41; see also, Sambrook, 2001, Molecular Cloning: A Laboratory Manuel, 3^{rd} ed. (Cold Spring Harbor Laboratory Press, Cold Spring Harbor, N.Y.).

Screening for multiple genes in samples of genomic material according to the methods of the present invention, is generally carried out using arrays of oligonucleotide probes. These arrays may generally be “tiled” for a large number of specific genes. By “tiling” is generally meant the synthesis of a defined set of oligonucleotide probes which is made up of a sequence complementary to the target sequence of interest, as well as pre-selected variations of that sequence, e.g., substitution of one or more given positions with one or more members of the basic set of monomers. i.e. nucleotides. Tiling strategies are discussed in detail in Published PCT Application No. WO 95/11995, incorporated herein by reference in its entirety for all purposes. By “target sequence” is meant a sequence which has been identified as encoding a biomarker of interest or portion thereof, a related polymorphism or mutation (e.g., a single-base polymorphism also referred to as a “biallelic base”) of one of the identified biomarkers. It will be understood that the term “target sequence” is intended to encompass the various forms present in a particular sample of genomic material, i.e., both alleles in a diploid genome.

In a particular aspect, arrays are tiled for a number of specific, identified biomarker sequences. In particular, the array is tiled to include a number of detection blocks, each detection block being specific for a particular biomarker or set of biomarkers. For example, a detection block may be tiled to include a number of probes which span the sequence segment that includes a specific biomarker or a polymorphism thereof. To ensure probes that are complementary to each variant, the probes are synthesized in pairs differing, for example, at the biallelic base.

In addition to the probes differing at the biallelic bases, monosubstituted probes can be generally tiled within the detection block. These monosubstituted probes have up to a certain number of bases in either direction from the polymorphisms, substituted with the remaining nucleotides (selected from A, T, G, C or U). Typically, the probes in a tiled detection block will include substitutions of the sequence positions up to and including those that are 5 bases away from the base that corresponds to the polymorphism. Preferably, bases up to and including those in positions 2 bases from the polymorphism will be substituted. The monosubstituted probes provide internal controls for the tiled array, to distinguish actual hybridization from artifactual cross-hybridization.

A variety of tiling configurations may also be employed to ensure optimal discrimination of perfectly hybridizing probes. For example, a detection block may be tiled to provide probes having optimal hybridization intensities with minimal cross-hybridization. For example, where a sequence downstream from a polymorphic base is G C rich, it could potentially give rise to a higher level of cross-hybridization or “noise,” when analyzed. Accordingly, one can tile the detection block to take advantage of more of the upstream sequence.

Optimal tiling configurations may be determined for any particular biomarker or polymorphism by comparative analysis. For example, triplet or larger detection blocks may be readily employed to select such optimal tiling strategies.

Additionally, arrays will generally be tiled to provide for ease of reading and analysis. For example, the probes tiled within a detection block will generally be arranged so that reading across a detection block the probes are tiled in succession, i.e., progressing along the target sequence one or more nucleotides at a time.

Once an array is appropriately tiled for a given biomarker and related polymorphism or set of polymorphisms, the target nucleic acid is hybridized with the array and scanned. A target nucleic acid sequence, which includes one or more previously identified biomarkers, is amplified by well known amplification techniques, e.g., polymerase chain reaction (PCR). Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single biomarker, in the preferred aspects, the arrays of the invention will include multiple detection blocks, and thus be capable of analyzing multiple, specific biomarkers. For example, preferred arrays will generally include from about 50 to about 4,000 different detection blocks with particularly preferred arrays including from 10 to 3,000 different detection blocks.

In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G C rich stretches of a genomic sequence, separately from those falling in A T rich segments. This allows for the separate optimization of hybridization conditions for each situation.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

More broadly, the present invention provides a method of detecting a nucleic acid in a sample. The method comprises attaching a nucleic acid targeting moiety to a SCN. The nucleic acid targeting moiety comprises a sequence that binds to the nucleic acid of interest in the sample. The method comprises contacting a nucleic acid of interest with a SCN conjugate comprising a water-soluble SCN and a targeting moiety. The targeting moiety of the conjugate specifically binds to the nucleic acid. Then, the method comprises photobleaching the sample and detecting the remaining luminescence. The detection of residual fluorescence in the sample is a proxy for the conjugate bound to the nucleic acid in the sample.

The present invention also provides a method whereby two or more different target molecules and/or two or more regions on a given target molecule can be simultaneously detected in a sample. The method involves using a set of SCN conjugates, wherein each of the conjugates in the set has a differently sized SCN or a SCN of different composition attached to a targeting moiety that specifically binds to a different target molecule or a different region on a given target molecule in the sample. Preferably, the SCN of the conjugates range in size from 2 nm to 6.5 nm, which sizes allow the emission of luminescence in the range of blue to red. The SCN size that corresponds to a particular color emission is well-known in the art. Within this size range, any size variation of SCN can be used as long as the differently sized SCN can be excited at a single wavelength and differences in the luminescence between the differently sized SCN can be detected. Desirably, the differently sized SCN have a capping layer that has a narrow and symmetric emission peak. Preferably, the differently sized SCN have an inorganic capping layer that matches the structure of the core. More preferably, the differently sized s have a ZnS or a CdSe capping layer. Similarly, SCN of different composition or configuration will vary with respect to particular color emission. Any variation of composition between SCN can be used as long as the SCN differing in composition can be excited at a single wavelength and differences in the luminescence between the SCN of different composition can be detected. Detection of the different target molecules in the sample arises from the emission of multicolored luminescence generated by the SCN differing in composition or the differently sized SCN of which the set of conjugates is comprised. This method also enables different functional domains of a single protein, for example, to be distinguished.

Accordingly, the present invention provides a method of simultaneously detecting two or more different target molecules and/or two or more regions of a given target molecule in a sample. The method comprises contacting the sample with two or more conjugates of a water-soluble SCN and a targeting moiety, wherein each of the two or more conjugates comprises a SCN of a different size or composition and a targeting moiety that specifically binds to a different molecule or a different region of a given target molecule in the sample. The method further comprises detecting luminescence, wherein the detection of luminescence of a given color is indicative of a conjugate binding to a molecule in the sample.

In accordance with the present invention, two or more proteins or fragments thereof can be simultaneously detected in a sample. Alternatively, two or more nucleic acids can be simultaneously detected. In this regard, a sample can comprise a mixture of nucleic acids and proteins (or fragments thereof).

Preferably, in the method of detecting two or more target proteins or fragments thereof, the targeting moiety of each of the conjugates is a protein or a fragment thereof, such as an antibody or an antigenically reactive fragment thereof, and the target proteins or fragments thereof in the sample are antigens or epitopes thereof that are bound by the antibody or the antigenically reactive fragment thereof. Alternatively and also preferably, the targeting moietys of each of the conjugates is an antigen or epitope thereof and the proteins or fragments thereof in the sample are antibodies or antigenically reactive fragments thereof that bind to the antigen or epitope thereof. Also preferably, the targeting moiety of each of the conjugates is a nucleic acid and the proteins or fragments thereof in the sample are nucleic acid binding proteins, e.g., DNA binding proteins.

Also, in accordance with the present invention, two or more target nucleic acids can be simultaneously detected in a sample. Any of the above-described methods for detecting a target nucleic acid in a sample can be used with two or more conjugates comprising differently sized SCN attached to targeting moieties that can bind to target nucleic acids. Accordingly, one method of simultaneously detecting two or more nucleic acids in a sample comprises contacting the sample with two or more SCN-targetting moiety conjugates, in which each conjugate comprises a differently sized SCN attached to a targeting moiety, preferably a nucleic acid, in particular a single-stranded nucleic acid, or a protein or fragment thereof, such as a DNA binding protein, that specifically binds to a target nucleic acid in the sample; photobleaching the sample, and detecting luminescence, wherein the detection of luminescence of a given color indicates that a conjugate bound to its target nucleic acid in the sample.

Yet another method of simultaneously detecting two or more target nucleic acids in a sample involves using the above-described method, wherein the target nucleic acids to be detected are attached to a solid support of the kind described above, in accordance with the described methods for attaching a nucleic acid in a sample and the described methods for detecting said nucleic acid as set forth above.

In another embodiment of the inventive method of simultaneously detecting two or more target molecules in a sample, the sample comprises at least one nucleic acid and at least one protein or fragment thereof. The simultaneous detection of a target nucleic acid and a target protein or fragment thereof in a sample can be accomplished using the methods described above in accordance with the described methods for detecting a target protein or fragment thereof in a sample and the described methods for detecting a target nucleic acid in a sample as set forth above.

The above described conjugates and methods can be adapted for use in numerous other methods and biological systems to effect the detection of a target molecule. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. The present invention also has broad application for the real-time observation of cellular mechanisms in living cells, e.g. ligand-receptor interaction and molecular trafficking, due to the increased photostability of the SCN.

Any methods available in the art for identification or detection of a protein or polynucleotide of interest are encompassed herein. Methods for detecting a molecule of interest comprise any method that determines the quantity or the presence of the molecule of interest either at the nucleic acid or protein level.

### Diagnostic Assays

The present invention has application in various diagnostic assays, including, but not limited to, the detection of viral infection, cancer, cardiac disease, liver disease, genetic diseases, and immunological diseases. The present invention can be used in a diagnostic assay to detect certain viruses, such as HIV and Hepatitis, by, for example, removing a sample to be tested from a patient; contacting the sample with a water-soluble SCN target moiety conjugate, wherein the targeting moiety is an antibody or antigenically reactive fragment thereof that binds to the virus; photobleaching the sample; and detecting the luminescence, wherein the detection of luminescence indicates that the virus is present in the sample. The patient sample can be a bodily fluid, such as saliva, tears, blood, serum or urine. For example, an antibody to HIV gp 120 can be used to detect the presence of HIV in a sample; alternatively, HIV gp 120 can be used to detect the presence of antibodies to HIV in a sample.

The present invention also can be used in a diagnostic assay to determine ultra-low-level viral loads of certain viruses, such as HIV and Hepatitis, by detecting the viral nucleic acid. Determining the viral load of a patient is useful in instances where the number of viral particles is below the detection limits of current techniques. For example, this technique can be particularly useful for tracking ultra-low HIV levels in AIDS patients during advanced drug treatment, such as triple drug therapy, in which the viral load of the patient has been greatly reduced. The detection of viral nucleic acid can be accomplished by, for example, removing a sample to be tested from a patient; treating the sample to release the viral DNA or RNA; contacting the sample with a water-soluble SCN biomolecular conjugate, wherein the targeting moiety binds to the nucleic acid of the virus; photobleaching the sample; and detecting the luminescence, wherein the detection of luminescence indicates that the virus is present in the sample.

Using this method, the detection of viral nucleic acid is accomplished by removing a sample to be tested from a patient; treating the sample to release the viral DNA or RNA; attaching capture probes to a solid support, wherein the capture probes comprise a sequence that binds to the viral nucleic acid in the sample; contacting the attached capture probes with the viral nucleic acid, thereby immobilizing the viral nucleic acid on the solid support; contacting the immobilized viral nucleic acid with a SCN conjugate, wherein the targeting moiety of the conjugate specifically binds to the viral nucleic acid; photobleaching the sample; and detecting luminescence, wherein the detection of luminescence indicates that the conjugate bound to the viral nucleic acid in the sample.

Preferably, the solid support is a glass surface, a transparent polymer surface, a membrane, or the like, to which the capture probe can be attached. The capture probe can be any molecule that is capable of both attaching to the solid support surface and binding to the target viral nucleic acid. Preferably, the capture probe is a single-stranded oligonucleotide comprising a first nucleic acid sequence that binds to a complementary sequence attached to the solid support and a second nucleic acid sequence that binds to a third nucleic acid sequence in the viral genome. The oligonucleotide comprising the first and second nucleic acid sequences can have a length of about 20 to 50 bases. Preferably, the oligonucleotide has a length of at least about 30 bases. Desirably, the third nucleic acid sequence in the viral genome is a conserved sequence.

The SCN conjugate comprises a SCN attached to a targeting moiety that specifically binds to the third sequence of the target viral nucleic acid in a region other than that which is bound by the second sequence of capture probe sequence. The targeting moiety can be any molecule that can bind to the target viral nucleic acid. Preferably, the targeting moiety is an oligonucleotide that contains a fourth sequence that is complementary to the third sequence in the target viral genome. Alternatively, the targeting moiety can be a DNA binding protein that binds specifically to the target viral nucleic acid.

In addition to the detection of a single virus, the present invention can be used to detect simultaneously the viral load of various types of viruses or the viral load of various sub-types of a single virus by detecting the different species of viral nucleic acid. One method of simultaneously detecting multiple viral nucleic acids in a sample comprises contacting the sample with a set of conjugates, wherein each conjugate of the set comprises a differently sized SCN attached to a probe targeting moiety that specifically binds to a target viral nucleic acid in the sample; and detecting the multicolored luminescence, wherein the detection of multicolored luminescence indicates that each of the differently conjugates bound to its target viral nucleic acid in the sample.

The present invention can be used in a similar manner to detect certain disease states, such as, for example, cancer, cardiac disease or liver disease, by removing a sample to be tested from a patient; contacting the sample with a water-soluble SCN biomolecular conjugate, wherein the targeting moiety is an antibody or antigenically reactive fragment thereof that binds to a protein associated with a given disease state, wherein the disease is, for example, cancer, cardiac disease or liver disease; Photobleaching the sample; and detecting the luminescence, wherein the detection of luminescence indicates the existence of a given disease state. In these cases, the sample can be a cell or tissue biopsy or a bodily fluid, such as blood, serum or urine. The protein can be a marker or enzyme associated with a given disease, the detection of which indicates the existence of a given disease state. The detection of a disease state can be either quantitative, as in the detection of an over- or under-production of a protein, or qualitative, as in the detection of a non-wild-type (mutated or truncated) form of the protein. In regard to quantitative measurements, preferably the luminescence of the SCN conjugate-target protein complex is compared to a suitable set of standards. A suitable set of standards comprises, for example, the SCN conjugate of the present invention in contact with various, predetermined concentrations of the target being detected. One of ordinary skill in the art will appreciate that an estimate of, for example, amount of protein in a sample, can be determined by comparison of the luminescence of the sample and the luminescence of the appropriate standards.

The present invention also can be used to detect a disease state, such as a genetic disease or cancer, by removing a sample to be tested from a patient; contacting the sample with water-soluble SCN biomolecular conjugate, wherein the targeting moiety is a nucleic acid that specifically hybridizes with a nucleic acid of interest; Photobleaching the sample; and detecting the luminescence, wherein the detection of luminescence indicates the existence of a given disease state. In these cases, the sample can be a derived from a cell, tissue or bodily fluid. The gene of interest can be a marker for a disease-state, such as BRCAI, which may indicate the presence of breast cancer.

The above-described methods also can be adapted for in vivo testing in an animal. The conjugate should be administered to the animal in a biologically acceptable carrier. The route of administration should be one that achieves contact between the conjugate and the targeting moiety, e.g., protein or nucleic acid, to be assayed. The in vivo applications are limited only by the means of detecting luminescence. In other words, the site of contact between the conjugate and the biomolecule to be assayed must be accessible by a luminescence detection means. In this regard, fiber optics can be used. Fiber optics enable light emission and detection as needed in the context of the present inventive methods.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Hippocampal Cultures

Primary cultures of hippocampal neurons from E19 rat embryos were plated on glass coverslips at 100,000 per ml in Neuralbasal media with B27 supplements (Sigma). Hippocampal neurons were dissociated in L-15 media with collagenase (20 mg/ml, Sigma) and dispase (96 mg/ml, Sigma). Enzymatic digestion was carried out at 37° C. for ˜45 min and cells triturated periodically with a fire polished pipette to facilitate dissociation. Neurons were washed twice in 1×PBS (Gibco) and plated on poly-D-lysine (Sigma) coated covers slips in Neurobasal media (Gibco). Neurons were maintained at 37° C. with 5% CO2 and used 10 to 14 days after isolation.

### Antibodies

The primary antibodies used were a polyclonal BKCa channel (Alomone Labs) at 1:150 and monoclonal MAP2 (a gift of V. Lee) at 1:250. The secondary antibodies used were anti-rabbit Alexa 546 and anti-mouse Alexa 488 at 1:400 (Molecular Probes). Anti-digoxigenin Fab Fragments conjugated to Qdot 565 (Invitrogen) or alkaline phosphatase (Roche) were used at 1:250. AlexaFluor 488 phalloidin (Molecular Probes) was used according to the manufactures protocol at 1:40.

### Immunocytochemistry

Primary rat hippocampal neurons were fixed on glass coverslips 10-14 days after plating, permeabilized with 0.3% TritonX-100, and processed for staining. Neurons were blocked at room temperature for 60 min in 3% bovine serum albumin, 1×PBS and 0.1% Tween-20. The primary and secondary antibodies were diluted in the blocking solution. The neurons were washed with 1×PBS with 0.1% Tween-20. Images were visualized with an Olympus Fluoview 1000 confocal scan head. For each cell, five randomly placed line scans were taken from three separate regions of interest for each dendritic segment and analyzed with Metamorph image processing software.

### ISH Using Cultured Hippocampal Neurons

Antisense digoxigenin-labeled KCNMA1 RNA probes (350 to 550 bp) were generated by in vitro transcription. Two separate, non-overlapping probes against i16 transcripts were used with equal success (data not shown). Primary neurons (10 to 14 days) were fixed in 4% paraformaldehyde and permeabolized with 0.3% TritonX-100. Cells were prehybridized at 42° C. for ˜4 h with 50% formamide, 1′ Denhardt's solution, 4′ SSC, 10 mM DTT, 0.1% CHAPS, 0.1% Tween-20, 500 mg/ml yeast tRNA, and 500 mg/ml salmon sperm DNA. Hybridization was performed at 42° C. for ˜16 h with 15 ng/ml probe in prehybridization buffer with the addition of 8% Dextran sulfate. Anti-digoxigenin F_{ab} fragments conjugated to Qdot 565 were used for detection (Invitrogen). The samples were subjected to photobleaching and the Qdot signal was detected under an Olympus Fluoview 1000 confocal scan head attached on inverted microscope. The samples were subjected to photobleaching to remove background autofluorescence before Qdot signal detection under an Olympus Fluoview 1000 confocal scan head attached on inverted microscope. After photobleaching, images were captured with a 458-nm excitation laser, and emissions were collected by spectral detector range of 550-594 nm spectrum. All images were captured with same parameters. Metamorph software was used to process images with same settings otherwise noted on the figure legend. For these ISH, we have used two procedures: one based upon alkaline phosphatase and nitro-blue tetrazolium (NBT)/5′-bromo-4-chloro-3′-indolyphosphate (BCIP) detection and a second procedure based upon fluorescent Qdot detection of RNAs. The first procedure is used to provide a good detail of cellular morphology in comparison to the mRNA signal detected with alkaline phosphatase and NBT and BCIP. In our hands, conventional protocols for fluorescent ISH did not yield a reproducibly consistent signal for the low-abundance BK_{Ca} channel variant mRNA ISH. A preimaging photobleaching process eliminates endogenous autofluorescence and in combination with the robust stability and lack of photobleaching of Qdots permits low level signal to be detected.

### Confocal Imaging and Data Analysis

ISH samples were subjected to photobleaching by exposure to a full spectral scan for two seconds to remove background autofluorescence before Qdot signal detection under an Olympus Fluoview 1000 confocal scan head. Since sources of autofluorescence vary depending on the origin and subsequent processing of a given biological samples, one skilled in the art would appreciate that the duration of photobleaching required for a given sample may require routine optimization. After photobleaching, images were captured with 458 nm excitation laser and emissions were collected by spectral detector range of 550-594 nm spectrum. All the images were captured with same parameters. The Metamorph image processing program was used to process images with same settings. Line scan analysis for BKCa channel protein distribution was performed after image acquisition. From whole cell images, region of interest were selected based on MAP2 staining and a random 1×25 pixel line scan area perpendicular to MAP2 orientation was used to obtain intensity profiles for MAP2 and BKCa channel signal. These data are presented as fluorescence intensity as a function of distance from the center of the MAP2 signal. For spine head analysis (FIG. 6), a 25 pixel round region of interest was randomly assigned in the phalloidin image channel and fluorescence intensities from other channels was measured. Statistical test was performed by Sigmaplot program. At least 2 different batches of cell culture were used for each experiment.

### Preparing SCN

A SCN or quantum dot associated with a targeting moiety is incubated in a solution with high protein content prior to addition to sections, blots, cells or other biological samples. This solution can be 10% BSA, 5% dissolved dried milk, or other such protein solutions.

The results of the experiments presented in this Example are now described.

### Example 1

### Signal Intensity of Alexa 488 and Qdot-565 Before and After Photobleaching

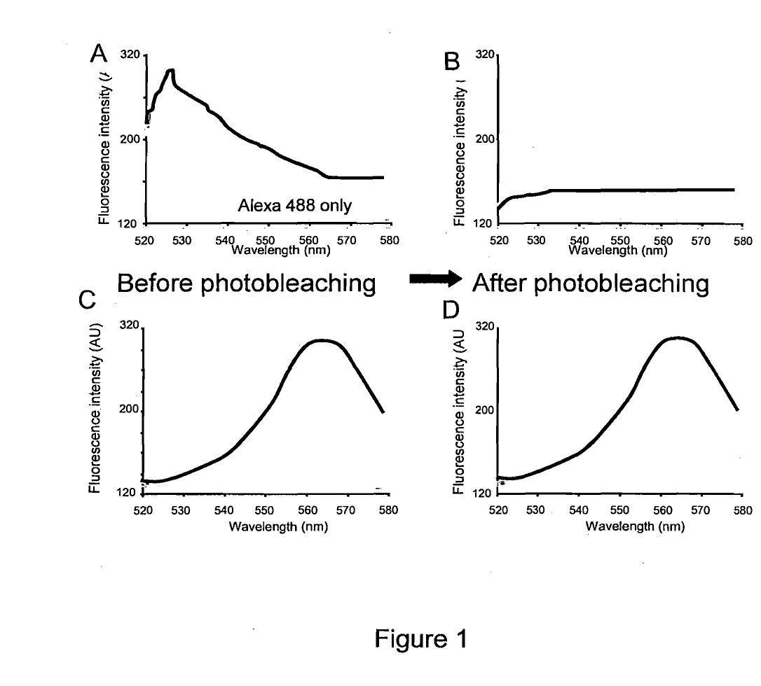

Primary rat hippocampal neurons were prepared for immunohistochemistry. Anti-digoxigenin Fab garments conjugated to Qdot 565 (Invitrogen) were used at 1:250. AlexaFluor 488 phalloidin (Molecular Probes) was used at 1:40. Images were taken with an Olympus Fluoview 1000 confocal scan head. For each cell, five randomly placed line scans were taken from three separate regions of interest and analyzed with the Metamorph image processing software. In FIG. 1, the emission spectral signature over the range from 520 to 580 nm wavelength was obtained before and after full spectral photobleaching of a sample for two seconds. In a sample stained only for Alexa 488, the photobleaching procedure abolishes Alexa 488 signal (FIG. 1B). In a second sample stained with Qdot-565, the identical full-spectrum photobleaching protocol was used, however the Qdot-565 signal was not affected (FIG. 1C and FIG. 1D).

### Example 2

### Selective Elimination of Unwanted Fluorescent Signals in a Single Sample Using Photobleaching

In order to confirm the specificity of the photobleaching technique and establish the utility of the present invention for use with multiple probes in a single sample, Qdot-565 and Alexa 488 were applied to a single sample at concentration of 1:250 and 1:40 respectively. The sample was photobleached using a full spectral scan for two seconds. The sample was then spectrally scanned and fluorescent intensity measured before and after photobleaching. Before photobleaching. The solid line shows the emission spectrum resulting from 458 nm excitation. The dotted line shows the remaining emission spectrum following photobleaching. These date clearly demonstrate the elimination of unwanted fluorescence signal of specific wavelength with sparing of the Qdot signal.

### Example 3

### In Situ Hybridization Using Cultured Hippocampal Neurons

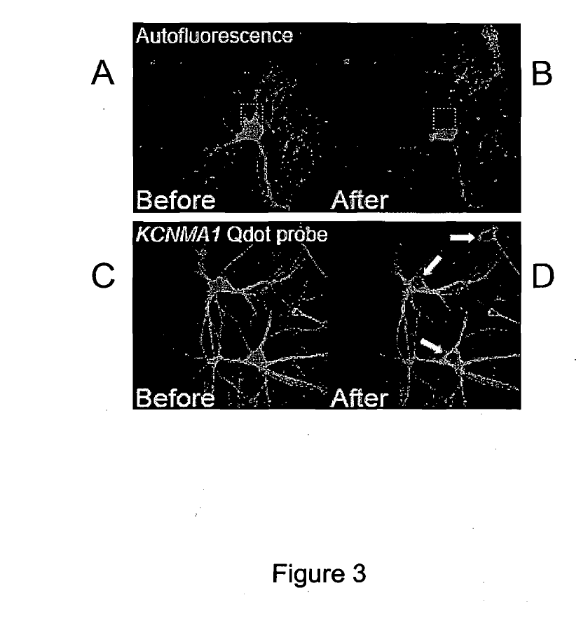

Antisense digoxigenin-labeled KCNMA1 RNA probes (350 to 550 bp) were generated by in vitro transcription. Two separate, non-overlapping probes against i16 transcripts were used with equal success (data not shown). Primary rat hippocampal cultures (10 to 14 days) were fixed for 15 minutes in 4% paraformaldehyde at room temperature, washed in 1×PBS and permeabolized with 1×PBS and 0.3% TritonX-100. Cells were prehybridized at 42° C. for ˜4 hours with 50% formamide, 1×Denhardt's solution, 4×SSC, 10 mM DTT, 0.1% CHAPS, 0.1% Tween-20, 500 ug/ul yeast tRNA and 500 ug/ul salmon sperm DNA. Hybridization was performed at 42° C. for ˜16 hours with 15 ng/ul probe in prehybridization buffer with the addition of 8% Dextran sulfate. Anti-digoxigenin Fab fragments conjugated to Qdot 565 were used for detection. The samples were subjected to photobleaching to remove background autofluorescence before Qdot signal detection under an Olympus Fluoview 1000 confocal scan head attached on inverted microscope. After photobleaching, images were captured with 458 nm excitation laser and emissions were collected by spectral detector range of 550-594 nm spectrum. All the images were captured with same parameters. The metamorph image processing program were used to process images with same settings otherwise noted on the figure legend. For these ISH, we have used two procedures: one based upon alkaline phosphatase and nitro-blue tetrazolium (NBT)/5′-bromo-4-chloro-3′-indolyphosphate (BCIP) idetection and a second procedure based upon fluorescent Qdot detection of RNAs. The first procedure is utilized in FIG. 3 to provide a good detail of cellular morphology in comparison to the mRNA signal detected with alkaline phosphatase and NET and BCIP. Conventional protocols for fluorescent ISH did not yield a reproducibly consistent signal for the low-abundance BKCa channel variant mRNA ISH. A pre-imaging photobleaching process eliminates endogenous autofluorescence and in combination with the robust stability and lack of photobleaching of Qdots permits low level signal to be detected. The limit of resolution for this technique is the detection of a single SCN. The optimization of the procedure is presented in FIG. 1.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of detecting a protein moiety in a biological sample, said method comprising contacting said protein with a fluorescent semiconductor nanocrystal (SCN), photobleaching said sample to reduce unwanted fluorescence, and detecting said fluorescent SCN, wherein said SCN comprises a modification comprising a targeting moiety.

2. The method of claim 1, wherein said biological sample is selected from a tissue, a cell, a biopsy, and a body sample.

3. The method of claim 1, wherein said fluorescent semiconductor nanocrystal is water soluble.

4. The method of claim 1, wherein said targeting moiety specifically binds to said protein.

5. The method of claim 4, wherein said targeting moiety comprises an antibody directed against said protein, or fragment thereof.

6. The method of claim 1, wherein said targeting moiety comprises an antibody and further wherein said method comprises an immunoassay selected from the group consisting of Western blot, ELISA, immunopercipitation, immunohistochemistry, immunofluorescence, radioimmunoassay, dot blotting, and FACS.

7. The method of claim 1, wherein said SCN is conjugated to streptavidin.

8. The method of claim 1, wherein said SCN is conjugated to a secondary antibody comprising the F(ab')₂ fragment of affinity purified antibodies cross adsorbed against serum proteins from a mammal.

9. The method of claim 8, wherein said mammal is selected from the group consisting of a human, a rat, a mouse, a rabbit, and a goat.

10. The method of claim 1, wherein said SCN is conjugated to a secondary antibody comprising the F(ab')₂ fragment of affinity purified antibodies cross adsorbed against serum proteins from a non-mammal.

11. The method of claim 10, wherein said non-mammal is a chicken.

12. The method of claim 1, wherein said SCN emits light with a characteristic wavelength of 450-495 nm.

13. The method of claim 1, wherein said SCN emits light with a characteristic wavelength of 495-570 nm.

14. The method of claim 1, wherein said SCN emits light with a characteristic wavelength of 570-590 nm.

15. The method of claim 1, wherein said SCN emits light with a characteristic wavelength of 590-620 nm.

16. The method of claim 1, wherein said SCN emits light with a characteristic wavelength of 620-750 nm.

17. A method of detecting a polynucleotide moiety in a biological sample, said method comprising contacting said polynucleotide with a fluorescent SCN, photobleaching said sample to reduce unwanted fluorescence, and detecting said SCN, wherein said SCN comprises a modification comprising a targeting moiety.

18. The method of claim 17, wherein said biological sample is selected from a tissue, a cell, a biopsy, and a body sample.

19. The method of claim 17, wherein said SCN is water soluble.

20. The method of claim 17, wherein said targeting moiety specifically binds to said polynucleotide moiety.

21. The method of claim 17, wherein detection of said polynucleotide comprises a nucleic acid assay selected from the group consisting of a Northern blot, a Southern blot, in situ hybridization, a PCR assay, an RT-PCR assay, a probe array, a gene chip, and a microarray.

22. A method of detecting a biomolecule moiety of interest in a biological sample, said method comprising contacting said biomolecule with a fluorescent SCN, photobleaching said sample to reduce unwanted fluorescence, and detecting said SCN, wherein said SCN comprises a modification comprising a targeting moiety.

23. The method of claim 22, wherein said biological sample is selected from a tissue, a cell, a biopsy, and a body sample.

24. The method of claim 22, wherein said SCN is water soluble.

25. The method of claim 22, wherein said targeting moiety specifically binds said biomolecule of interest.

26. The method of claim 22, wherein said method comprises an immunoassay selected from the group consisting of Western blot, ELISA, immunopercipitation, immunohistochemistry, immunofluorescence, radioimmunoassay, dot blotting, and FACS.

27. The method of claim 22, wherein detection of said polynucleotide comprises a nucleic acid assay selected from the group consisting of a Northern blot, a Southern blot, in situ hybridization, a PCR assay, an RT-PCR assay, a probe array, a gene chip, and a microarray.
